(19) United States
(12) Patent Application Publication
(10) Pub. No.: US 2007/0255178 A1
(43) Pub. Date: Nov. 1, 2007

(54) Method for lancing a target site in low ambient light conditions

(75) Inventors: Manuel Alvarez-Icaza, Inverness (GB); Damian Edward Haydon Baskeyfield, Auldearn (GB)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: LifeScan Scotland, Ltd.

(21) Appl. No.: 11/509,949

(22) Filed: Aug. 24, 2006

Related U.S. Application Data
(60) Provisional application No. 60/796,344, filed on Apr. 27, 2006.

(51) Int. Cl. A61B 5/00

(52) U.S. Cl. 600/583

(57) ABSTRACT

A method for lancing a target site in low ambient light conditions includes employing diffuse light produced by an integrated light source of a lancing device to achieve user spatial awareness in a low ambient light condition. The method further includes illuminating a target site, also in the low ambient light condition, with an intense beam of light from the integrated light source and urging the lancing device against the target site. Subsequently, the target site is lanced with a lancet held by the lancing device.

### BACKGROUND OF THE INVENTION

1. Field of the Invention

The present invention relates, in general, to medical devices and, in particular, to lancing devices and their associated methods.

2. Description of the Related Art

Conventional lancing devices generally have a rigid housing, various operating mechanisms (also referred to collectively as a lancing mechanism) and a lancet that can be armed and launched so as to briefly protrude from one end of the lancing device. For example, conventional lancing devices can include a lancet that is mounted within a rigid housing such that the lancet is movable relative to the rigid housing along a longitudinal axis thereof. Typically, the lancet is spring-loaded and is launched upon release of the spring to penetrate (i.e., “lance”) a target site (e.g., a dermal tissue target site). A bodily fluid sample (e.g., a whole blood sample) can then be expressed from the penetrated target site for collection and analysis. Conventional lancing devices are described, for example, in U.S. Pat. No. 5,730,753 to Morita, U.S. Pat. No. 6,045,567 to Taylor et al., U.S. Pat. No. 6,071,250 to Douglas et al., U.S. Pat. No. 6,156,051 to Schraga, U.S. Pat. No. 6,197,040 to LeVaughn et al., and U.S. Pat. No. 6,607,543 to Purcell et al., each of which is hereby fully incorporated by reference.

Conventional lancing devices typically require a user to arm the lancing device, urge the lancing device against a target site, and then press a button or other switch to manually activate the lancing device such that a lancet within the device is launched (also referred to as “fired”) towards the target site. The lancet then penetrates (e.g., lances) the target site, thereby creating an opening for the expression of a bodily fluid sample.

Moreover, conventional lancing devices often include a cap with a distal end that engages the target site during use. Such a cap usually has an aperture (i.e., opening), through which the lancet protrudes during use. After a cap is engaged (i.e., contacted) with a target site, the lancet is launched to penetrate the target site. A biological fluid sample, typically blood, is thereafter expressed from the lanced target site. The expressed biological fluid sample can then, for example, be tested for an analyte (such as glucose, lactate, ketones and HbA1c) using an associated meter. Such testing (also referred to as “analysis”) can include, for example, determination of the presence and/or concentration of an analyte in the expressed biological fluid sample.

The aforementioned user is typically in possession of a portable, compact and discrete blood glucose monitoring kit that includes, in addition to the lancing device, various ancillary items such as a meter, a supply of control solution, a sterile lancet supply, alcohol swabs, an owner's booklet and quick reference guide, a registration card, spare batteries and a carrying case. A user may desire to perform bodily fluid collection, analysis and documentation (i.e. perform a “test”) by employing the lancing device and other items in the blood glucose monitoring kit under a variety of settings, including settings with low ambient light conditions.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings, of which:

FIG. 1 is a simplified perspective view of a conventional lancing device;

FIG. 2 is a simplified side view of a lancing device with integrated light source according to an exemplary embodiment of the present invention;

FIG. 3 is a simplified diagram of a circuit for a light source that is suitable for use in various embodiments of the present invention;

FIG. 4A is a simplified side view of a lancing device according to another exemplary embodiment of the present invention;

FIG. 4B is a simplified side view of the lancing device of FIG. 4A positioned on a surface;

FIG. 4C is a simplified bottom view of a portion of the lancing device of FIG. 4A with a stand thereof in a fold-away position;

FIG. 4D is a simplified end view of a portion of the lancing device of FIG. 4A with a stand thereof in a fold-away position;

FIG. 4E is a simplified end view of a portion of the lancing device of FIG. 4A with a stand thereof in a deployed position;

FIG. 5 is a simplified side view of a lancing device according to yet another exemplary embodiment of the present invention;

FIG. 6 is a simplified, perspective, exploded view of the lancing device of FIG. 5;

FIG. 7 is a simplified side view of a cap with integrated light source according to an exemplary embodiment of the present invention;

FIG. 8 is a simplified cross-sectional side view of a cap with integrated light source according to another exemplary embodiment of the present invention;

FIG. 9 is a simplified side view of a lancing device with integrated light source according to a further exemplary embodiment of the present invention;

FIG. 10 is a simplified perspective view of a lancing device with integrated light source according to a still further exemplary embodiment of the present invention;

FIG. 11 is a simplified block diagram of a lancing system according to an exemplary embodiment of the present invention; and

FIG. 12 is a flow diagram depicting stages in a process for lancing a target site in low ambient light conditions according to an exemplary embodiment of the present invention.

### DETAILED DESCRIPTION OF EXAMPLARY EMBODIMENTS OF THE INVENTION

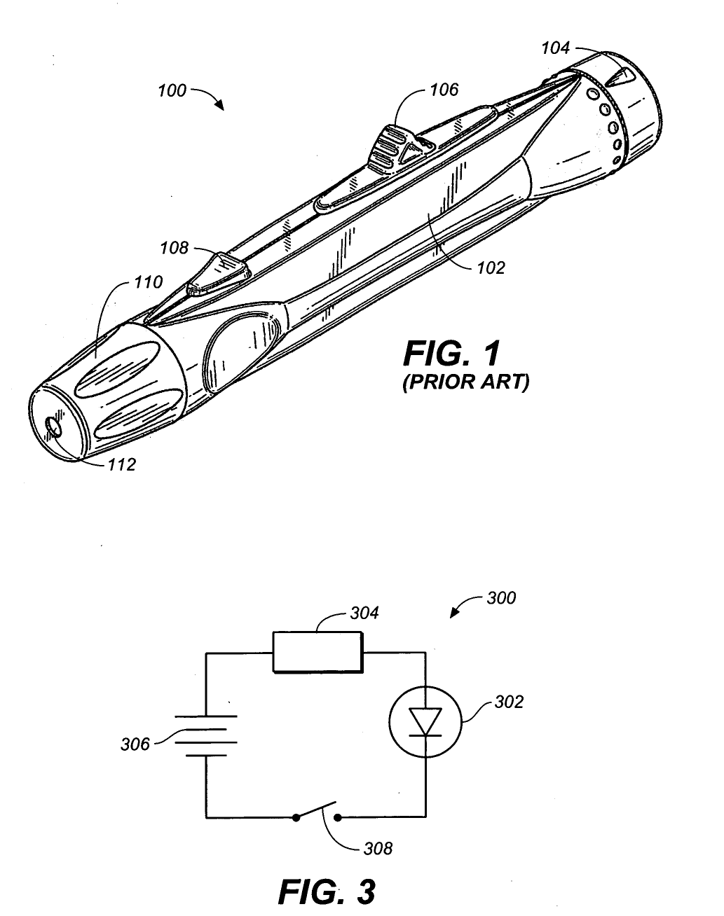

FIG. 1 is a simplified perspective view of a conventional lancing device 100. Lancing device 100 includes a housing 102, a depth-setting gauge 104, an arming member 106, a trigger button 108 and a cap 110. Furthermore, cap 110 includes an opening (aperture) 112 through which a lancet (not shown in FIG. 1) protrudes during use of lancing device 100.

Although not depicted in FIG. 1, conventional lancing device 100 also includes a lancing mechanism disposed at least partially within housing 102 and configured to lance a target site (such as a dermal tissue target site) with the lancet. Such a lancing mechanism can include, for example, a moveable lancet holder disposed within the housing and configured to hold the lancet, a launching mechanism also disposed within the housing, and an arming mechanism and a trigger mechanism both disposed partially within the housing. Moreover, the moveable lancet holder, launching mechanism, arming mechanism and trigger mechanism are operatively connected to lance a target site with a lancet. A non-limiting example of a commercially available conventional lancing device is the OneTouch® UltraSoft™ Adjustable Blood Sampler available from LifeScan Inc., Milpitas, USA. Further descriptions of conventional lancing devices are in, for example, U.S. Pat. Nos. 6,045,567 and 6,197,040, as well as U.S. Design Pat. No. USD428150, each of which is incorporated fully herein by reference.

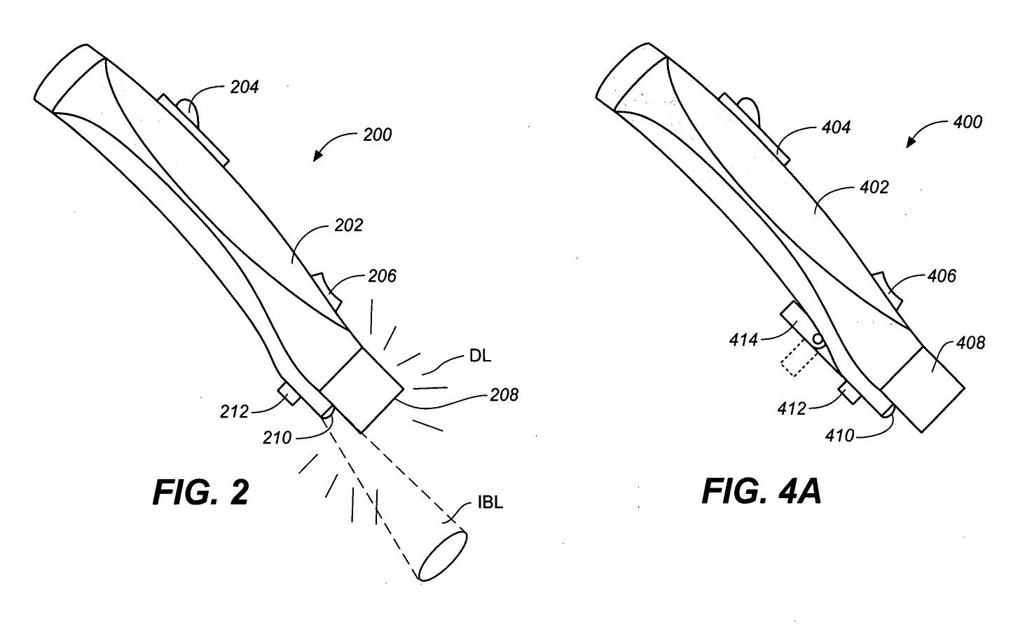

FIG. 2 is a simplified perspective view of a lancing device 200 with integrated light source according to an exemplary embodiment of the present invention. FIG. 3 is a simplified diagram of a circuit 300 for a light source suitable for use in various embodiments of the present invention, including lancing device 200.

Lancing device 200 includes a housing 202, a lancing mechanism (of which arming member 204 and trigger button 206 are depicted) disposed at least partially within housing 202 and configured to lance a target site with a lancet (not shown). Lancing device 200 also includes a cap 208 and an integrated light source 210. Cap 208 can be formed as an integral portion of housing 202, as a removable portion of housing 202 or as an independent component of lancing device 200.

Light source 210 includes a light source switch 212. Although the embodiment of FIG. 2 depicts light source switch 212 in the form of a button, other suitable means of switching the light source on and off can be employed. For example, a light source switch can be configured such that twisting a portion of housing 202 (e.g., a knob located at the distal end of housing 202) serves to turn integrated light source 210 on and off.

Integrated light source 210, in combination with at least one of housing 202 and cap 208, produces an intense beam of light (IBL) for illumination of at least the target site and a weaker diffuse light (DL) that provides a user with spatial awareness while conducting a test. The intense beam of light (IBL) can be, for example, projecting essentially parallel to the longitudinal axis of the lancing device axis (as depicted in FIG. 2) to readily illuminate a target site during testing.

A non-limiting example of an intense beam of light (IBL) is a beam with a 15 degree angle cone and 900 Lux Illuminance at a distance of 0.1 m from the lancing device. In general, the Illuminance range for the intense beam of light can be, for example, in the range of 500 Lux to 2000 Lux for distances of between 5 cm and 15 cm from the lancing device. The Illuminance of the weaker diffuse light can be, for example, in the range of 3 Lux to 10 Lux for distances in the range of 5 cm to 15 cm from the lancing device.

Light sources employed in embodiments of the present invention can produce light of any suitable wavelength or wavelengths. Since the human eye has heightened sensitivity to light of a wavelength near 550 nm it can, for example, be suitable for a light source to employ (i) a green Light Emitting Diode (LED) that emits light with a wavelength of approximately 530 nm or (ii) a yellow LED that emits light with a wavelength of approximately 585 nm. However, a white LED that emits light across a range of wavelengths that appears natural to a user may also be suitable. The selection of a suitable light source can also be based on other relevant concerns such as the light source's required operating power.

Referring to FIG. 3 in particular, light source 210 can, for example, include a high intensity light emitting diode (LED) 302, a resistor 304, a battery 306 and a circuit switch 308. Battery 306 and resistor 304 can, for example, be disposed at least partially within housing 202. In the embodiment of FIG. 3, battery 306 is dedicated to powering circuit 300 and is, therefore, independent from any power source for an associated meter or any other power source related to testing. This dedication enables functioning of the meter to be uncompromised by use of the integrated light source and potential exhaustion of battery 306.

Illustrative but non-limiting examples of suitable components for a circuit as depicted in FIG. 3 are (i) a yellow LED (e.g., yellow LED part number LYK376 commercially available from Osram with a forward voltage of 2V and typical current of 20 mA), a 3V battery (e.g., a CR1220 lithium magnesium dioxide battery commercially available from Panasonic) and a 50 ohm resistor; and (ii) a white LED (e.g., white LED part number NSPW300BS commercially available from Nichia with a forward voltage of 3.6V and a typical current of 20 mA), two 3V batteries in series and a 120 ohm resistor.

As would be apparent to one skilled in the art once apprised of the present disclosure, circuit switch 308 is operatively connected to light source switch 212 such that a user can manually activate integrated light source 210. In addition, if desired, circuit 300 can include a timing element (i.e., a timer, not shown) configured to limit the duration of time during which integrated light source 210 produces the intense beam of light IBL and diffuse light DL. Such a timing element can serve to conserve battery power in the event a user does not turn off integrated light source 210 using light source switch 212 once a test has been completed. For example, upon activation of integrated light source 210 by a user via light source switch 212, the timing element may automatically activate high intensity LED 302 for a predetermined duration of time that is sufficient to complete a successful test (for example, a duration of time of approximately 3 minutes). Power consumption can also be conserved by employing a rapidly pulsing light source (e.g., a light emitting diode (LED) pulsing at a frequency of 50 Hz or greater), the pulsing of which will not be visible to the human eye. The rapid pulsing can be achieved by adjusting the LED's duty cycle, thus conserving power.

The certain types of light created by integrated light source 210 (i.e., both an intense beam of light and a diffuse light) are particularly beneficial when a test is being conducted in low ambient light conditions (for example, light conditions of 5 Lux or less). For example, the intense beam of light can be employed to illuminate an area to locate ancillary items required to perform the test, to illuminate a target site, while the diffuse light can be employed provide a user with general spatial awareness. Since the light source is integrated with the lancing device (for example, integrated with a lancing device's housing or cap), a user has the convenience of not carrying a separate light source such as a flashlight.

In the embodiment of FIG. 2, the intense beam of light (IBL) is produced by integrated light source 210 as a slightly diverging conical beam of light transmitting from the distal end of lancing device 200. Moreover, cap 208 is formed of a transparent material (e.g., a transparent polypropylene material) and operatively positioned with respect to integrated light source 210 such that diffuse light DL is produced around the circumference of cap 208 (see FIG. 2).

Once apprised of the present disclosure, one skilled in the art will, recognize that the diffuse light can be produced by, for example, employing a cap with a surface finish, surface ribs or surface ridges on all or a portion of the cap. Such surface finishes, ridges or ribs can be predetermined to provide an angle of incidence that permits light to escape. In alternative embodiments, the lancing device housing can be similarly configured for light escape and the production of diffuse light.

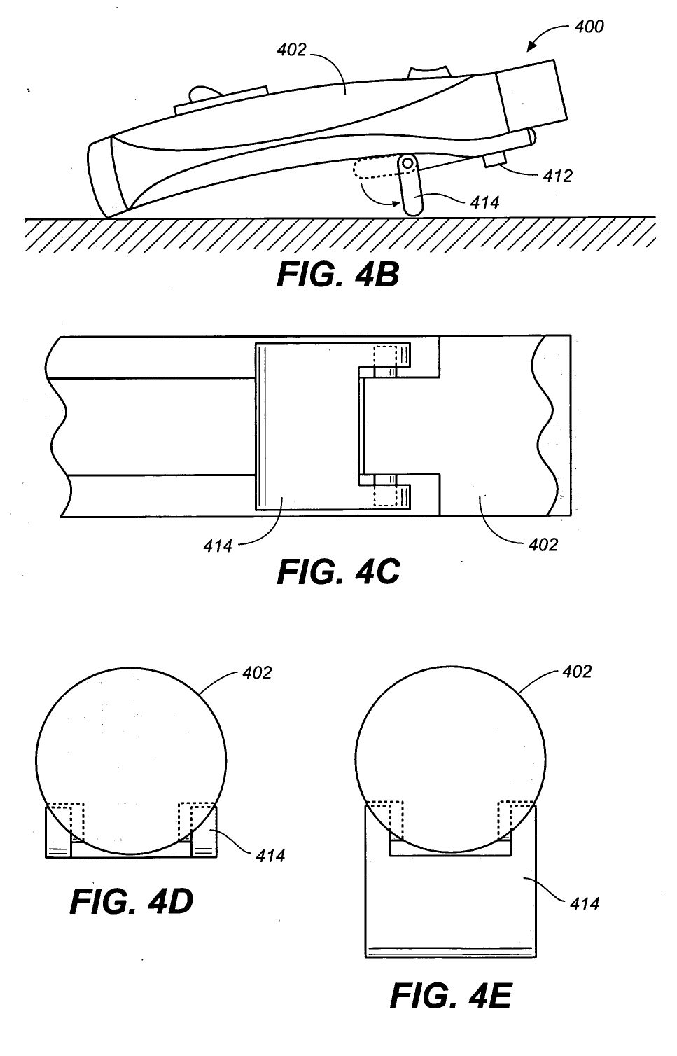

FIGS. 4A-4E are various simplified views of a lancing device 400 with integrated light source according to another exemplary embodiment of the present invention. Lancing device 400 includes a housing 402, a lancing mechanism (of which arming member 404 and trigger button 406 are depicted) disposed at least partially within housing 402 and configured to lance a target site with a lancet (not shown). Lancing device 400 also includes a cap 408, and an integrated light source 410 with light source switch 412. Lancing device 400 also includes a stand 414. Stand 414 can be placed in a foldaway position (depicted with solid line in FIG. 4A) or a deployed position (depicted with dashed lines in FIG. 4A).

Integrated light source 410, in combination with at least one of housing 402 and cap 408, produces an intense beam of light (IBL, not shown in FIGS. 4A-4E) for illumination of, for example, a target site or ancillary items, as well as a weaker diffuse light (DL, not shown in FIGS. 4A-4E) that provides a user with spatial awareness while conducting a test. The intense beam of light (IBL) can, for example, be essentially parallel to the longitudinal axis of the lancing device axis of lancing device to readily illuminate a target site during testing.

Any suitable technique can be employed to product the intense beam of light. However, such an intense beam of light (IBL) can be produced, for example, by configuring the housing and/or cap such that light from the integrated light source reaches the housing (or cap) surface at an angle that is nearly normal to the surface, thus enabling the housing (or cap) to focus the light into an intense beam of light (IBL). In this circumstance, it is beneficial for the housing (or cap) can be formed of a transparent material, such as polymethylmethacrylate, with an index of refraction that is greater than the index of refraction of air.

Stand 414 is configured to enable a user to employ integrated light source 410 in a hands-free manner. When stand 414 is in a deployed position, lancing device 400 can be securely placed on a surface in a stable manner to provide the user with light in which to work (see FIGS. 4B and 4E in particular). When not in use, stand 414 can be placed in the fold-away position (see FIGS. 4C and 4D in particular).

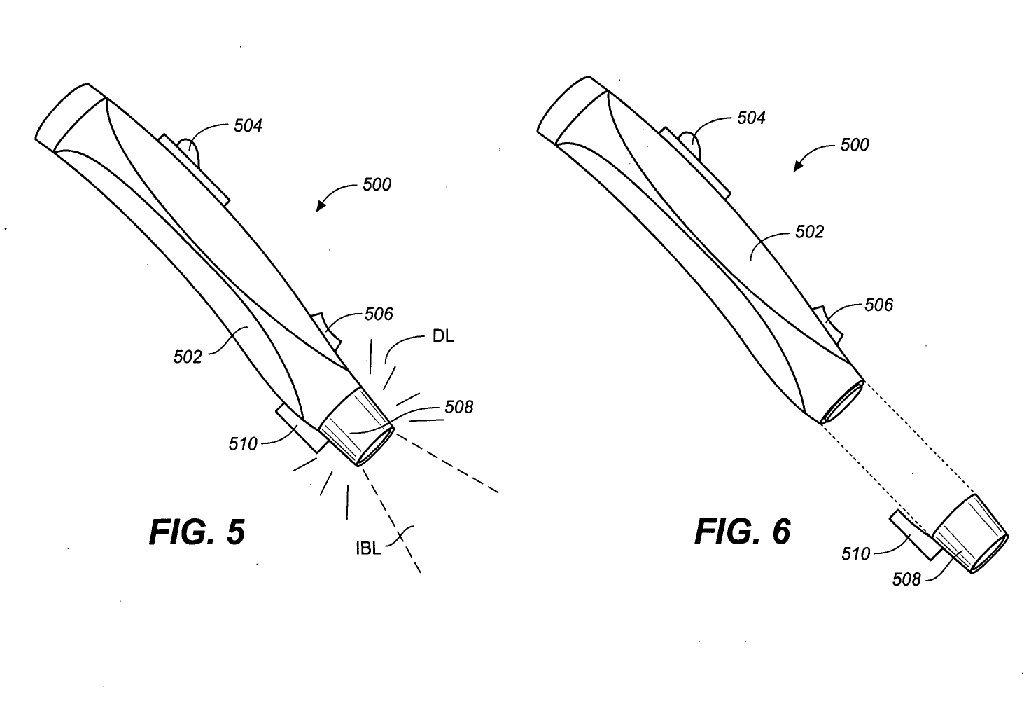

FIGS. 5 and 6 are simplified depictions of a lancing device 500 according to still another exemplary embodiment of the present invention. Lancing device 500 includes a housing 502, a lancing mechanism (of which arming member 504 and trigger button 506 are depicted) disposed at least partially within housing 502 and configured to lance a target site with a lancet (not shown). Lancing device 500 also includes a removable cap 508 and an integrated light source 510. In the embodiment of FIGS. 5 and 6, integrated light source 510 is integrated into removable cap 508 (see FIG. 6 in particular).

Integrated light source 510, in combination with removable cap 508, produces an intense beam of light (IBL) for illumination of at least the target site and a weaker diffuse light (DL) that provides a user with spatial awareness while conducting a test. The intense beam of light (IBL) can, for example, be essentially parallel to the longitudinal axis of the lancing device axis of lancing device (as depicted in FIG. 5) to readily illuminate a target site during testing. In the embodiment of FIG. 5, removable cap 508 is configured to channel the intense beam of light along the longitudinal axis of lancing device 500 and is at least partially formed of a transparent material to produce diffuse light DL.

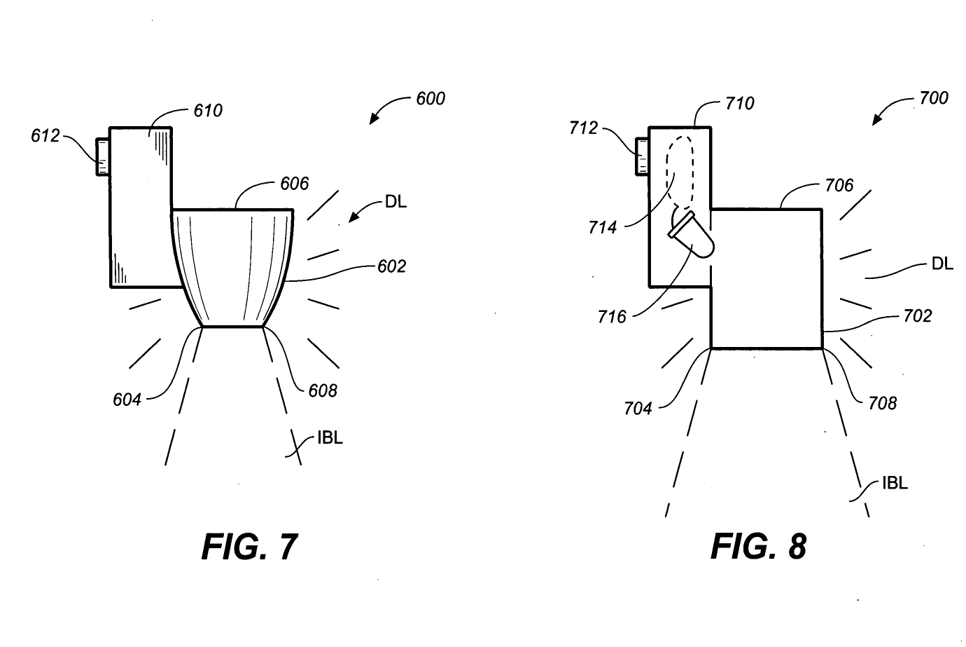

FIG. 7 is a simplified side view of a cap 600 with integrated light source according to an exemplary embodiment of the present invention. Cap 600 includes a cap body 602 with a distal end 604 and a proximal end 606. Distal end 604 includes a target site contact surface 608. Proximal end 606 can be configured for attachment to a lancing device (for example, to a housing of a lancing device) by a snap fit, frictional fit or other suitable attachment technique.

Cap 600 also includes an integrated light source 610 with light source switch 612. Integrated light source 610, in combination with cap body 602, produces an intense beam of light (IBL) for illumination of, for example, a target site and a weaker diffuse light (DL) that provides a user with spatial awareness while conducting a test. In this regard, cap body 602 serves as a light guide channeling photons emitted from integrated light source 610 into intense beam of light IBL.

FIG. 8 is a simplified cross-sectional side view of a cap 700 with integrated light source according to another exemplary embodiment of the present invention. Cap 700 includes a cap body 702 with a distal end 704 and a proximal end 706. Distal end 704 includes a target site contact surface 708. Proximal end 706 can be configured for attachment to a lancing device (for example, to a housing of a lancing device) by a snap fit, frictional fit or other suitable attachment technique.

Cap 700 also includes an integrated light source 710 with light source switch 712, battery 714 and light emitting diode 716. Light source 710, in combination with cap body 702, produces an intense beam of light (IBL) for illumination of, for example, a target site and a weaker diffuse light (DL) that provides a user with spatial awareness while conducting a test. In this regard, cap body 702 serves as a light guide channeling photons emitted from integrated light source 710 into intense beam of light IBL.

Integrated light source 710 can be positioned at any suitable angle with respect to cap body 702 such that a diffuse light DL and an intense beam of light IBL are formed. For example, integrated light source 710 can be positioned to optimize photon capture within cap body 702, thereby channeling the photons to form a focused beam of intense beam of light IBL.

Once apprised of the present disclosure, one skilled in the art will recognize that light sources employed in embodiments of the present invention light source can be of any suitable type including, for example, LED light sources. LED light sources can include a coupled light guide, for example a fiber optic light guide, resulting in a fiber optic light source. A fiber optic light source can, for example, be molded within a housing or a cap body of a removable cap during manufacture in a manner which optimizes light capture therein, and guiding the light to form an intense beam of light and an area of diffuse light.

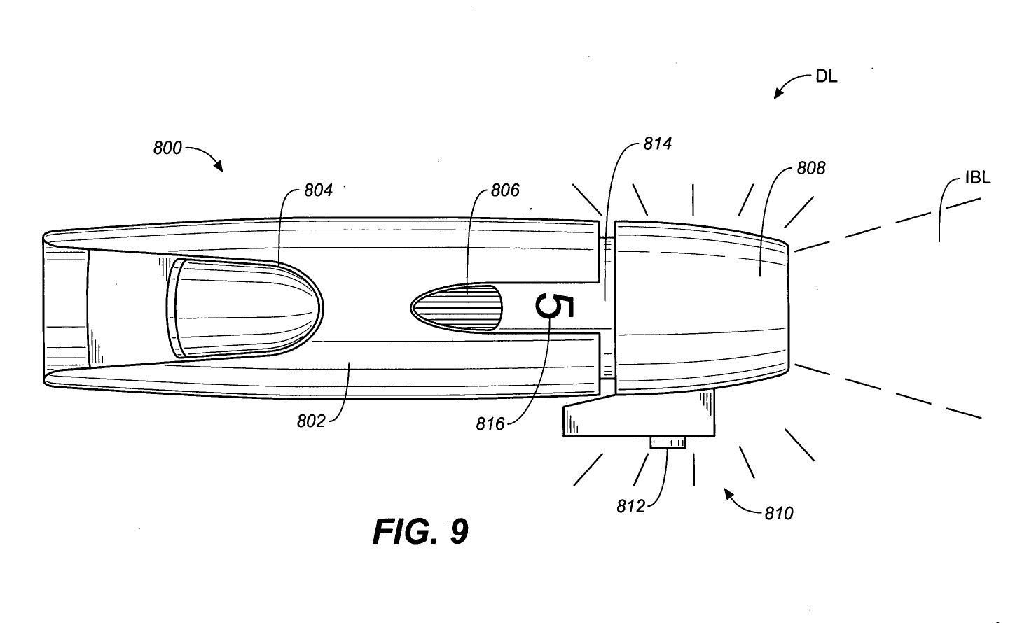

FIG. 9 is a simplified side view of a lancing device 800 with integrated light source according to a further exemplary embodiment of the present invention. Lancing device 800 includes a housing 802, a lancing mechanism (of which arming member 804 and trigger button 806 are depicted) disposed at least partially within housing 802 and configured to lance a target site with a lancet (not shown). Lancing device 800 also includes a cap 808 and an integrated light source 810. Integrated light source 810 includes a light source switch 812.

Integrated light source 810, in combination with cap 808, produces an intense beam of light (IBL) for illumination and a weaker diffuse light (DL) that provides a user with spatial awareness while conducting a test. The intense beam of light (IBL) can, for example, be essentially parallel to the longitudinal axis of lancing device 800 (as depicted in FIG. 9) to readily illuminate a target site during testing.

Lancing device 800 also includes a depth setting gauge with background area 814 formed of light transmitting material and numerals 816 (of which only the numeral “5” is visible in the view of FIG. 9 for illustration and explanation purposes) formed of opaque material (such as black ink). Integrated light source 810 and the depth setting gauge are configured such that light is guided into background area 814 to beneficially reveal an appropriate depth setting numeral (e.g., the numeral “5” depicted in FIG. 9) to a user in low ambient light conditions. The light can be guided, for example, using a light guide component that employs total internal reflection as a means for guiding the light.

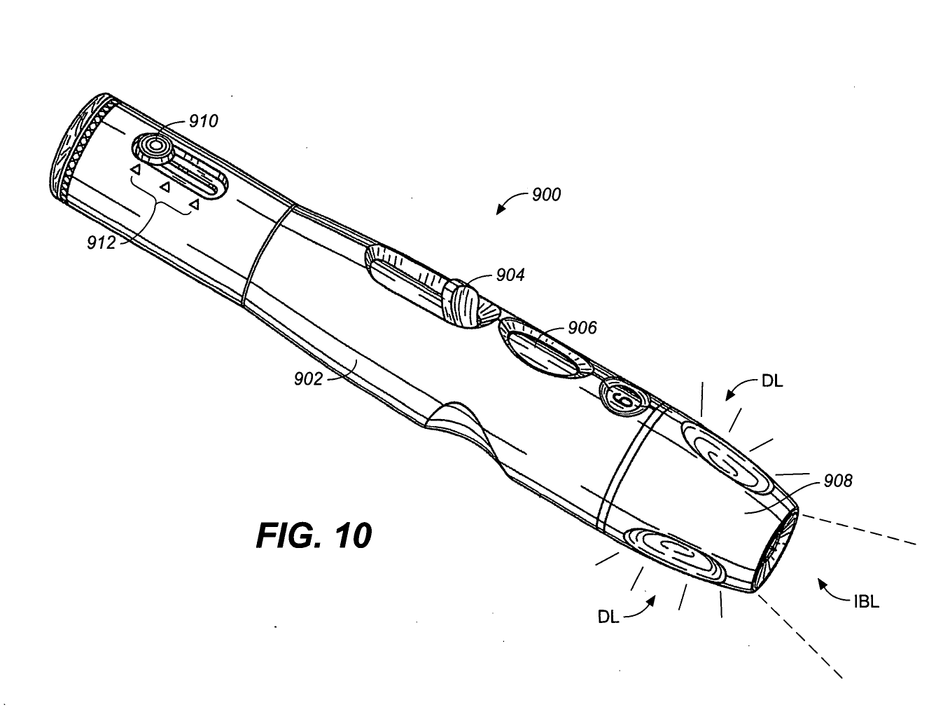

FIG. 10 is a simplified perspective view of a lancing device 900 with integrated light source according to a still further exemplary embodiment of the present invention. Lancing device 900 includes a housing 902, a lancing mechanism (of which arming member 904 and trigger button 906 are depicted) disposed at least partially within housing 902 and configured to lance a target site with a lancet (not shown). Lancing device 900 also includes a cap 908 and an integrated light source (not entirely visible in the view of FIG. 10).

The light source of lancing device 900 includes a light source switch 910 configured to activate the light source at three intensity settings (each marked by one of three arrows 912) as light source switch 910 is moved in the direction of arrow A by a user. A user can, thereby, adjust the intensity of the intense beam of light and the diffuse light to suit the low ambient light conditions present during testing.

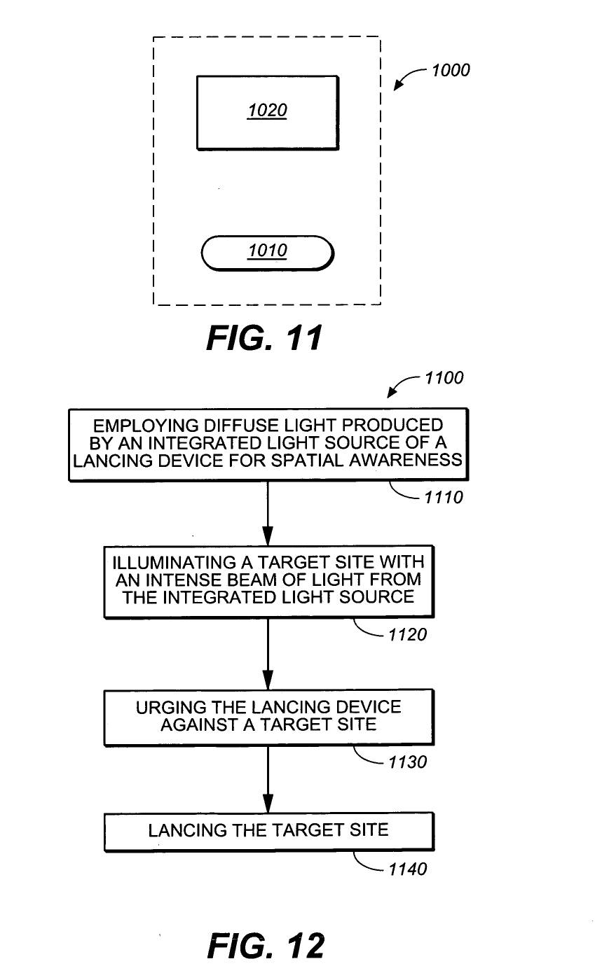

FIG. 11 is a simplified block diagram of a lancing system 1000 (within the boundaries of the dashed lines) according to an exemplary embodiment of the present invention. Lancing system 1000 includes a lancing device 1010 and a meter 1020.

Lancing device 1010 can be any suitable lancing device according to embodiments of the present invention and, therefore, includes (i) a housing, (ii) a lancing mechanism disposed at least partially within the housing and configured to lance a target site with a lancet, and (iii) an integrated light source. The light source, in combination with at least the housing, produces an intense beam of light for illumination and a diffuse light that provides a user with spatial awareness.

Meter 1020 is configured for the analysis of a bodily fluid sample expressed from a target site lanced by the lancing mechanism. For example, meter 1020 can be configured for the determination of blood glucose in a whole blood sample using techniques that are known to those of skill in the art.

FIG. 12 is a flow diagram depicting stages in a method 1100 for lancing a target site (e.g., a dermal tissue target site on a user's finger) in low ambient light conditions according to an exemplary embodiment of the present invention. Once apprised of the present disclosure, one skilled in the art will recognize that method 1100 can be, for example, accomplished using lancing devices, caps and systems according to various embodiments of the present invention and can include techniques associated with such lancing devices, caps and systems as described herein.

Method 1100 includes employing diffuse light produced by an integrated light source of a lancing device to achieve user spatial awareness in a low ambient light condition (see step 1110). A target site in the low ambient light condition is illuminated with an intense beam of light from the integrated light source, as set forth in step 1120. Subsequently, the lancing device is urged against the target site and the target site is lanced with a lancet held by the lancing device (see steps 1130 and 1140, respectively).

If desired in method 1100, the intense beam of light and/or the diffuse light can also be employed to locate, manipulate and employ items that are ancillary to lancing. Such ancillary items include, but are not limited to, a meter, a supply of control solution, a sterile lancet supply, alcohol swabs, test strip(s), and an owner's booklet and quick reference guide. For example, the intense beam of light and/or diffuse light can be employed as an aid in locating a test strip and applying a bodily fluid sample thereto.

Methods according to embodiments of the present invention beneficially enable a lancing device user to successfully and easily lance a target site under low ambient light conditions by providing both diffuse light for general special awareness and an intense beam of light for illumination of a target site.

It should be understood that various alternatives to the embodiments of the invention described herein may be employed in practicing the invention. It is intended that the following claims define the scope of the invention and that structures and methods within the scope of these claims and their equivalents be covered thereby.

What is claimed is:

1. A method for lancing a target site in low ambient light conditions, the method comprising:
employing diffuse light produced by an integrated light source of a lancing device to achieve user spatial awareness in a low ambient light condition;
illuminating a target site, in the low ambient light condition, with an intense beam of light from the integrated light source;
urging the lancing device against the target site; and
lancing the target site with a lancet held by the lancing device.

2. The method of claim 1 further including the step of locating blood glucose kit ancillary items with the aid of at least one of the intense beam of light and the diffuse light.

3. The method of claim 2 wherein the locating step occurs while the lancing device is positioned on a lancing device stand.

4. The method of claim 1 wherein further including, prior to the lancing step, a step of verifying a lancing device depth gauge setting that has been illuminated by the integrated light source.

5. The method of claim 1 further including, after the lancing step, the step of deactivating the light source via automatic operation of an integrated light source timer.

6. The method of claim 1 further including the step of using at least one the diffuse light and the intense beam of light to aid in the manipulation of at least one blood glucose kit ancillary item.

7. The method of claim 6 wherein the using step occurs while the lancing device is positioned on a lancing device stand.

8. The method of claim 1 further including the step of using at least one the diffuse light and the intense beam of light to aid in the employment of at least one blood glucose kit ancillary item.

9. The method of claim 8 wherein the using step occurs while the lancing device is positioned on a lancing device stand.

10. The method of claim 1 wherein the lancing step includes lancing a dermal tissue target site for the expression of a whole blood sample.
